(19) United States
(12) Patent Application Publication
(10) Pub. No.: US 2021/0363515 A1
(43) Pub. Date: Nov. 25, 2021

(54) HIGH THROUGHPUT METHOD OF PERSONALIZED TARGET ENRICHMENT PANEL ASSEMBLY

(71) Applicant: NOVA BIOLABS, INC., Longmont, CO (US)

(72) Inventors: Brady Patrick CULVER, Longmont, CO (US); Michael BANOS, Longmont, CO (US)

(21) Appl. No.: 16/980,721

(22) PCT Filed: Feb. 15, 2019
(86) PCT No.: PCT/US2019/018271

§ 371 Date: Sep. 14, 2020

Related U.S. Application Data
(60) Provisional application No. 62/631,125, filed on Feb. 15, 2018.
(60) Provisional application No. 62/768,480, filed on Nov. 16, 2018.

(51) Int. Cl. C12N 15/10

(52) U.S. Cl. CPC C12N 15/1058

(57) ABSTRACT

Disclosed herein are methods of generating a subpool of oligonucleotides from a larger pool of oligonucleotides. A subpool of oligonucleotides can contain a portion of a gene of interest, and can be generated on a subject by subject basis on demand. Also disclosed herein are methods of processing a subpool of oligonucleotides to generate probes for sequencing. Also disclosed herein are systems and methods for using subpools of oligonucleotides generated from a larger pool for high-throughput sequencing.

### CROSS-REFERENCE

This application claims priority to U.S. Provisional Application No. 62/631,125 filed on Feb. 15, 2018, and to U.S. Provisional Application No. 62/768,480 filed on Nov. 16, 2018, each of which is hereby incorporated by reference in their entirety.

### SUMMARY

Disclosed herein are methods of preparing a subpool of subject-specific oligonucleotides from a first pool based on a gene profile from a subject. A method can comprise obtaining a first pool, where a first pool can comprise a plurality of oligonucleotides. In some embodiments, a plurality of oligonucleotides can comprise a universal primer binding site. In some embodiments, each oligonucleotide of a plurality of oligonucleotides can have the same universal primer binding site. In some embodiments, a plurality of oligonucleotides can comprise a content region. In some embodiments, a content region can comprise an overlap region to a portion of a gene. In some embodiments, an overlap region can comprise at most two mismatches to a gene. In some embodiments, each oligonucleotide of the plurality of oligonucleotides can have a different content region. In some embodiments, a subset of oligonucleotides within a plurality of oligonucleotides can comprise a content region with at least a portion of a same gene. In some embodiments, a plurality of oligonucleotides can comprise a target-specific primer binding site flanking content region. In some embodiments, a method can further comprise amplifying exponentially from a first pool a subset of oligonucleotides using a primer pair. In some embodiments, a first primer of a primer pair can bind to a universal primer binding site. In some embodiments, a second primer of a primer pair can bind to a target-specific primer binding site to produce a subpool of subject-specific oligonucleotides. In some embodiments, a second primer can be chosen based on a gene profile for a subject. In some embodiments, an amplifying can produce oligonucleotides with a content region comprising genes specified in a gene profile; thereby preparing a subpool of subject-specific oligonucleotides. In some embodiments, subject-specific oligonucleotides can be target enrichment primers. In some embodiments, a first primer or a second primer can comprise a recognition element. In some embodiments, a recognition element can be biotin. In some embodiments, a content region can comprise a site specific endonuclease. In some embodiments, a method can further comprise digesting a subpool of subject-specific oligonucleotides with an ApoI restriction endonuclease to produce an ApoI-digested subpool. In some embodiments, a method can further comprise digesting a digested subpool to produce a single stranded capture probe. In some embodiments, a gene profile can be obtained from a subject sample. In some embodiments, a subject sample can be a solid biopsy. In some embodiments, a subject sample can be a liquid biopsy. In some embodiments, a subject sample can be a blood sample. In some embodiments, a gene profile can be obtained from a subject database. In some embodiments, a gene profile can comprise a portion of a gene. In some embodiments, a gene profile can comprise at least one full gene. In some embodiments, a gene profile can comprise at least one exon of a gene. In some embodiments, a gene profile can comprise at least 2 genes. In some embodiments, a database can comprise a gene profile from a plurality of subjects. In some embodiments, a first pool can be present on a microarray. In some embodiments, a plurality of oligonucleotides can comprise DNA. In some embodiments, a plurality of oligonucleotides can comprise RNA. In some embodiments, an amplifying can be accomplished using polymerase chain reaction. In some embodiments, an amplifying can produce oligonucleotides spanning at least a portion of a gene. In some embodiments, an amplifying can produce oligonucleotides spanning at least one full gene. In some embodiments, an amplifying can produce oligonucleotides spanning at least one exon of a gene. In some embodiments, an amplifying can produce oligonucleotides spanning at least 2 genes. In some embodiments, a gene profile can include a gene selected from the group consisting of: BRCA1, BRCA2, BARDI, TP53, BRAF, Myc, Bcl-2, CDKN1β, NOTCH1, EGFR, FGFR1, FGFR2, FGFR3, HNF1A, JAK1, JAK2, JAK3, KIT, KRAS, MET, SRC, and any combination thereof. In some embodiments, an oligonucleotide database can be consulted to select a second primer of a primer pair. In some embodiments, an oligonucleotide database can comprise instructions for locating a second primer of a primer pair. In some embodiments, an oligonucleotide database can comprise an oligonucleotide sequence for a second primer of a primer pair.

Also disclosed herein are systems that can comprise a computer readable memory storing on an electronic storage device an oligonucleotide database. In some embodiments, a system can comprise a computer processor. In some embodiments, a computer processor can be configured to access an oligonucleotide database and select a primer pair. In some embodiments, a primer pair can comprise a first primer and a second primer from the oligonucleotide database. In some embodiments, a primer pair can be capable of amplifying each of a set of genes from a gene profile after a set of genes is input into a system.

Also disclosed herein are kits that can comprise a first oligonucleotide pool. In some embodiments, a first oligonucleotide pool can comprise a plurality of oligonucleotide. In some embodiments, each oligonucleotide can comprise a universal primer binding site. In some embodiments, each oligonucleotide of a plurality of oligonucleotides can have the same universal primer binding site. In some embodiments, a first oligonucleotide pool can comprise a content region. In some embodiments, a content region can comprise at least a portion of a gene. In some embodiments, each oligonucleotide of a plurality of oligonucleotides can have a different content region. In some embodiments, a plurality of genes can comprise BRCA1, BRCA2, BARDI, TP53, BRAF, Myc, Bcl-2, CDKN1β, NOTCH1, EGFR, FGFR1, FGFR2, FGFR3, HNF1A, JAK1, JAK2, JAK3, KIT, KRAS, MET, and SRC. In some embodiments, a first oligonucleotide pool can comprise a target-specific primer binding site. In some embodiments, oligonucleotides can comprise a content region with a portion of the same gene. In some embodiments, oligonucleotides comprising a content region with a portion of the same gene can have the same target-specific primer binding site.

Also disclosed herein are methods that can comprise obtaining a subpool of subject-specific oligonucleotides as described herein. In some embodiments, a method can comprise contacting a subpool of subject-specific oligonucleotides with a subject sample. In some embodiments, a method can comprise performing a sequencing reaction.

Also disclosed herein are methods of monitoring a progression of a tumor. In some embodiments, a method can comprise obtaining a first pool of primers. In some embodiments, a first pool of primers can comprise a plurality of oligonucleotides. In some embodiments, each oligonucleotide can comprise a 5′ universal primer binding site. In some embodiments, each oligonucleotide of a plurality of oligonucleotides can have the same 5′ universal primer binding site. In some embodiments, each oligonucleotide can comprise a content region. In some embodiments, a content region can comprise at least a portion of a gene. In some embodiments, each oligonucleotide of a plurality of oligonucleotides can have a different content region. In some embodiments, a subset of oligonucleotides within a plurality of oligonucleotides can comprise a content region with at least a portion of a same gene. In some embodiments, each oligonucleotide can comprise a 3′ target-specific primer binding site. In some embodiments, a method can further comprise amplifying exponentially from a first pool a subset of oligonucleotides using a primer pair. In some embodiments, a first primer of a primer pair can bind to a 5′ universal primer binding site. In some embodiments, a second primer of a primer pair can bind to a 3′ target-specific primer binding site to produce a subpool of subject-specific oligonucleotides. In some embodiments, a method can further comprise contacting a polynucleotide isolated from a sample obtained from a subject with a subpool of subject-specific oligonucleotides. In some embodiments, a method can further comprise performing a sequencing reaction. In some embodiments, a method can further comprise repeating a step provided herein for a period of time, thereby monitoring a progression of a tumor.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts amplification of a subpool from an exemplary pool with 5′ and 3′ universal primer binding sites, and 5′ and 3′ content specific primer binding sites.

FIG. 2 depicts amplification of a subpool from an exemplary pool with 5′ and 3′ universal primer binding sites, and a 3′ content specific primer binding site.

FIG. 3 depicts amplification of a subpool from an exemplary pool with 5′ and 3′ universal primer binding sites, and a 5′ content specific primer binding site.

FIG. 4A-4B depict a method for construction of biotinylated sense hybrid capture probes from exemplary double stranded oligonucleotides from an exemplary pool with 5′ and 3′ universal primer binding sites, and a 3′ content specific primer binding site.

FIG. 5 depicts a method for construction of sense target enrichment primers from exemplary double stranded oligonucleotides using an exonuclease from a pool with 5′ and 3′ universal primer binding sites, and a 3′ content specific primer binding site.

FIG. 6A-6B depict a method for construction of biotinylated antisense hybrid capture probes from exemplary double stranded oligonucleotides from a pool with 5′ and 3′ universal primer binding sites, and a 3′ content specific primer binding site.

FIG. 7 depicts a method for construction of antisense target enrichment primers from exemplary double stranded oligonucleotides using an exonuclease from a pool with 5′ and 3′ universal primer binding sites, and a 3′ content specific primer binding site.

FIG. 8A-8B depict a method for construction of biotinylated antisense hybrid capture probes from exemplary double stranded oligonucleotides from a pool with 5′ and 3′ universal primer binding sites, and a 5′ content specific primer binding site.

FIG. 9 depicts a method for construction of antisense hybrid target enrichment primers from exemplary double stranded oligonucleotides using an exonuclease from a pool with 5′ and 3′ universal primer binding sites, and a 5′ content specific primer binding site.

FIG. 10A-10B depict a method for construction of biotinylated sense hybrid capture probes from exemplary double stranded oligonucleotides from a pool with 5′ and 3′ universal primer binding sites, and a 5′ content specific primer binding site.

FIG. 11 depicts a method for construction of sense target enrichment primers from exemplary double stranded oligonucleotides using an exonuclease from a pool with 5′ and 3′ universal primer binding sites, and a 5′ content specific primer binding site.

FIG. 12A depicts an exemplary subpool spanning genes of interest. FIG. 12B depicts a cartoon illustration of a subset of hybridization capture probes spanning exons in a gene locus.

FIG. 12C depicts a cartoon illustration of two subsets of hybridization capture probes spanning a gene locus. FIG. 12D depicts a cartoon illustration of a subset hybridization capture probes spanning exons across multiple gene loci.

FIG. 13 depicts amplification of a subset of a subpool from an exemplary pool with 5′ and 3′ universal primer binding sites, and a 3′ content specific primer binding site utilizing a modified 5′ universal primer.

FIG. 14 depicts amplification of a subset of a subpool from an exemplary pool with 5′ and 3′ universal primer binding sites, and a 3′ content specific primer binding site utilizing a modified 3′ content specific primer.

FIG. 15 depicts a method for construction of biotinylated sense RNA probes from exemplary double stranded oligonucleotides from an exemplary pool with 5′ and 3′ universal primer binding sites, and a 3′ content specific primer binding site

### DETAILED DESCRIPTION

Overview

Disclosed herein are methods for producing modular pools of DNA target enrichment primers or probes using a pre-designed pool of synthetic oligonucleotides as a feedstock. The pre-designed pool can include primer binding sites that can be used to strategically amplify a given subpool of primers on demand.

Also disclosed herein are methods of preparing probes (e.g. hybrid capture probes) from a subpool of primers. A set of probes prepared as described herein can be used to sequence one or more genes or portions thereof on a subject by subject basis.

Also disclosed herein are methods of using probes prepared from a subpool of primers to monitor the progression of a disease or condition on a subject by subject basis.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances a disease or condition can be cancer. A subject can be a patient. A subject can be an individual. In some instances, a subject, patient or individual can be used interchangeably.

In some instances, “treat,” “treating”, “treatment,” “ameliorate” or “ameliorating” and other grammatical equivalents can include prophylaxis. “Treat,” “treating”, “treatment,” “ameliorate” or “ameliorating” and other grammatical equivalents can further include achieving a therapeutic benefit and/or a prophylactic benefit. Therapeutic benefit can mean eradication of the underlying disease being treated. Also, a therapeutic benefit can be achieved with the eradication of one or more of the physiological symptoms associated with the underlying disease such that an improvement can be observed in a subject notwithstanding that, in some embodiments, the subject can still be afflicted with the underlying disease.

The term “nucleic acid,” as used herein, can refer to deoxyribonucleic acid (DNA), ribonucleic acid (RNA), or a variant thereof. A nucleic acid may include one or more subunits selected from adenosine (A), cytosine (C), guanine (G), thymine (T), and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that may be specific to one of more complementary A, C, G, T, or U, or complementary to a purine (i.e., A or G, or variant thereof) or pyrimidine (i.e., C, T, or U, or variant thereof). In some examples, a nucleic acid may be single-stranded or double stranded, in some cases, a nucleic acid is circular.

The terms “nucleic acid molecule” or “nucleic acid sequence,” as used herein, can refer to a polymeric form of nucleotides, or polynucleotide that may have various lengths, either deoxyribonucleotides (DNA) or ribonucleotides (RNA), or analogs thereof. The term “nucleic acid sequence” may refer to the alphabetical representation of a polynucleotide; alternatively, the term may be applied to the physical polynucleotide itself. This alphabetical representation can be input into databases in a computer having a central processing unit and used for mapping nucleic acid sequences or nucleic acid molecules to symbols, or bits, encoding digital information. Nucleic acid sequences or oligonucleotides may include one or more non-standard nucleotide(s), nucleotide analog(s) and/or modified nucleotides.

The term “oligonucleotide”, as used herein, can refer to a single-stranded nucleic acid sequence, and is typically composed of a specific sequence of four nucleotide bases: adenine (A); cytosine (C); guanine (G), and thymine (T) or uracil (U) when the polynucleotide is RNA.

Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methyl cytosine, 5-methyl cytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

The term “primer,” as used herein, can refer to a strand of nucleic acid that serves as a starting point for nucleic acid synthesis, such as polymerase chain reaction (PCR). In an example, during replication of a DNA sample, an enzyme that catalyzes replication starts replication at the 3′-end of a primer attached to the DNA sample and copies the opposite strand.

The term “probe” as used herein can refer to an oligonucleotide product produced by a method described herein. A “probe” can include a primer, a labeled hybridization probe as described herein, or an unlabeled hybridization probe as described herein. A probe can be single stranded, and can comprise a “sense” or an “antisense” portion of a gene of interest.

The term “polymerase” or “polymerase enzyme,” as used herein, can refer to any enzyme capable of catalyzing a polymerase reaction. Examples of polymerases include, without limitation, a nucleic acid polymerase. The polymerase can be naturally occurring or synthesized. An example polymerase is a Φ29 polymerase or derivative thereof. In some cases, a transcriptase or a ligase is used (i.e., enzymes which catalyze the formation of a bond) in conjunction with polymerases or as an alternative to polymerases to construct new nucleic acid sequences. Examples of polymerases include a DNA polymerase, a RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poc polymerase, Pab polymerase, Mth polymerase ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Pfutubo polymerase, Pyrobest polymerase, KOD polymerase, Bst polymerase, Sac polymerase, Klenow fragment polymerase with 3′ to 5′ exonuclease activity, and variants, modified products and derivatives thereof.

The term “pool” or “library” of oligonucleotides can be used interchangeably to refer to an initial feedstock of oligonucleotides. A pool can include DNA, RNA, or both. In some cases, a pool of DNA (e.g. cDNA) can be constructed by reverse transcribing RNA. In some embodiments, a pool of oligonucleotides can be present as either single stranded or double stranded nucleic acid. A pool of oligonucleotides as described herein can comprise a plurality of distinct oligonucleotides. In some instances, a pool may comprise from about 100 to about 1,000 distinct oligonucleotides, from about 100 to about 2,000 distinct oligonucleotides, from about 100 to about 3,000 distinct oligonucleotides, from about 100 to about 4,000 distinct oligonucleotides, from about 100 to about 5,000 distinct oligonucleotides, from about 100 to about 6,000 distinct oligonucleotides, from about 100 to about 7,000 distinct oligonucleotides, from about 100 to about 8,000 distinct oligonucleotides, from about 100 to about 9,000 distinct oligonucleotides, or from about 100 to about 10,000 distinct oligonucleotides. In some instances, a pool may comprise from about 1,000 to about 10,000 distinct oligonucleotides, from about 1,000 to about 20,000 distinct oligonucleotides, from about 1,000 to about 30,000 distinct oligonucleotides, from about 1,000 to about 40,000 distinct oligonucleotides, from about 1,000 to about 50,000 distinct oligonucleotides, from about 1,000 to about 60,000 distinct oligonucleotides, from about 1,000 to about 70,000 distinct oligonucleotides, from about 1,000 to about 80,000 distinct oligonucleotides, from about 1,000 to about 90,000 distinct oligonucleotides, or from about 1,000 to about 100,000 distinct oligonucleotides. In some instances, a pool may comprise from about 10,000 to about 100,000 distinct oligonucleotides, from about 10,000 to about 200,000 distinct oligonucleotides, from about 10,000 to about 300,000 distinct oligonucleotides, from about 10,000 to about 400,000 distinct oligonucleotides, from about 10,000 to about 500,000 distinct oligonucleotides, from about 10,000 to about 600,000 distinct oligonucleotides, from about 10,000 to about 700,000 distinct oligonucleotides, from about 10,000 to about 800,000 distinct oligonucleotides, from about 10,000 to about 900,000 distinct oligonucleotides, or from about 10,000 to about 1,000,000 distinct oligonucleotides.

The term “subpool” of oligonucleotides can refer to a set of oligonucleotides that can be recalled from the pool or library of oligonucleotides using a specific primer pair. The term “subset” or “subset of content” can refer to a portion of the subpool of oligonucleotides that may comprise a desired content region (e.g. a portion of the same gene). In some cases, a subpool of oligonucleotides with n subsets can be recalled from an initial pool of oligonucleotides by using n distinct primer pairs. In some cases, where n can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000.

The terms “universal” or “library” primer can be used interchangeable to refer primers that can anneal to a region that is common to all members of a pool of oligonucleotides. A universal or library primer can be on a 5′ end of an oligonucleotide, a 3′ end of an oligonucleotide, or both.

The terms “content-specific” or “target-specific” primer can be used interchangeable to refer primers that can anneal to a region that is common to members of a subpool of oligonucleotides that share a content region of interest. A content or target specific primer can flank a content region and can be on 5′ of the content region, 3′ of the content region, or both.

The term “content region” can refer to a portion of an oligonucleotide within a pool that can comprise a region of interest. In some instances, a content region can be a region that can anneal to a portion of a gene, including coding and non-coding regions. In some instances, a subpool of oligonucleotides can be recalled with a desired content region one or more content-specific or a target-specific primer pairs.

Amplification of Oligonucleotide Subpools

Disclosed herein are methods for amplifying a specific subpool of oligonucleotides from a pool of nucleotides on demand. A pool of oligonucleotides can be pre-designed to feature universal and identifying regions, and can contain desired content (e.g. a portion of a gene of interest) that can correspond to an identifying region. By properly designing a pool of oligonucleotides, a desired subpool of oligonucleotides with a desired content can be amplified and used for applications as described herein.

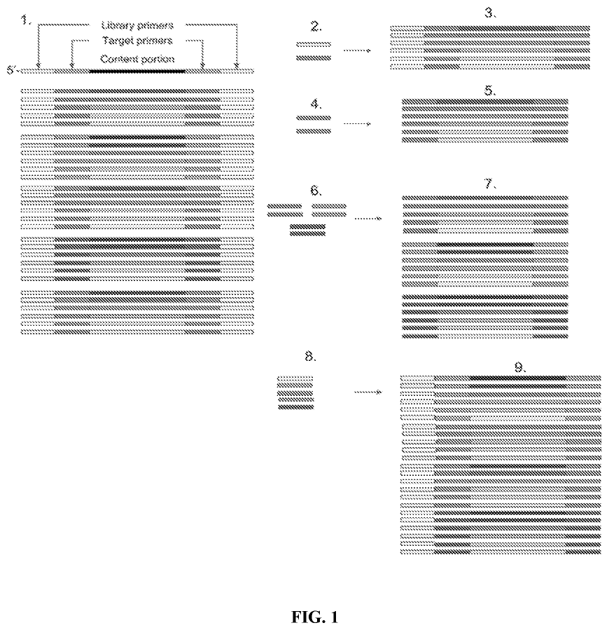

FIG. 1 depicts an exemplary construction of a first exemplary oligonucleotide pool 001, for which a subpool can be selectively amplified. Each molecule in the first exemplary pool of synthetic oligonucleotides can comprise in 5′ to 3′ order (left to right), a forward library primer sequence common to all molecules in the pool, a forward target specific primer sequence, a content portion, a reverse target specific primer binding site, and a reverse library primer binding site common to all molecules in the pool. Different pairs of target specific amplification primers in the oligonucleotide pool 001 are depicted in FIG. 1 with different shades. Different content sharing a same target specific primer pair is indicated in FIG. 1 by differing shades.

In embodiments, a plurality of distinct content sequences can be flanked by either the same forward target specific primer sequence or the same reverse target specific primer binding site or both of these elements, and can be referred to as a subset of content. Amplification with a forward universal primer and a single reverse target specific primer (primer pair 002) can recall a subpool 003 of oligonucleotide from the first pool of oligonucleotides 001. The subpool 003 produced using primer pair 002 and the first pool of synthetic oligonucleotides from 001 can comprise a common forward universal binding site, a common reverse target specific primer binding site, and content corresponding to the reverse target specific primer binding site.

Amplification with a forward target specific primer and reverse target specific primer (primer pair 004) can recall a subpool of 005 of oligonucleotides from the first pool of oligonucleotides 001. The subpool 005 produced using primer pair 004 and the first pool of synthetic oligonucleotides from 001 can comprise a common forward target specific primer binding site, a common reverse target specific primer binding site, and content corresponding to the forward or reverse target specific primer binding site. Oligonucleotides in subpool 005 can have the same content regions as oligonucleotides in subpool 003.

Amplification with three different forward target specific primers and three different reverse target specific primers (primer pairs 006) can be used to recall a subpool 007 with three distinct subsets of oligonucleotides 007 from the first pool of oligonucleotides 001. Each one of the three subsets in subpool 007 can comprise a common forward target specific primer binding site, a common reverse target specific primer binding site, and content corresponding to the forward or reverse target specific primer binding site.

In some embodiments, four distinct subsets of content can be amplified in the same PCR reaction from the first pool of oligonucleotides 001 using a forward library primer and a first reverse target specific primer, a second reverse target specific primer, a third reverse target specific primer, and a fourth reverse target specific primer in a reaction that can be referred to as a multiplex PCR. Amplification with a single forward universal primer and four distinct reverse target specific primers (primer pairs 008) can be used to recall a subpool of oligonucleotides 009 with four distinct subsets of sequences from the first pool of oligonucleotides 001. Each one of the four subsets in subpool 009 can comprise a common universal primer binding site, a common reverse target specific primer binding site, and content corresponding to the reverse target specific primer binding site.

In some embodiments, the content portion of the first pool of oligonucleotides 001 can be destined for use in target enrichment of massively parallel DNA sequencing libraries as hybridization capture probes. In some embodiments, the content portion of the first pool of synthetic oligonucleotides 001 can be destined for use in target enrichment of massively parallel DNA sequencing libraries as PCR primers.

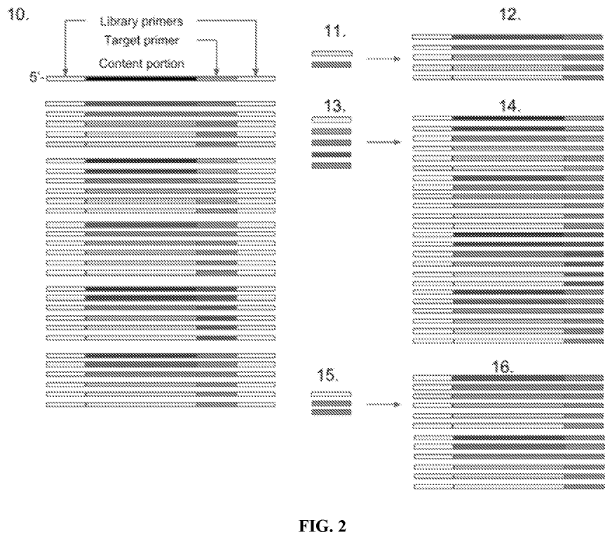

FIG. 2 depicts an exemplary construction of a second exemplary oligonucleotide pool 010, for which a subpool can be selectively amplified. Each molecule in the second exemplary pool 010 of oligonucleotides can comprise in 5′ to 3′ order (left to right), a forward library primer sequence common to all molecules in the pool, a content portion, a reverse target specific primer binding site, and a reverse library primer binding site common to all molecules in the pool. Different pairs of target specific amplification primers in the second oligonucleotide pool 010 are depicted in FIG. 2 with different shades. Different content sharing a same target specific primer pair is indicated in FIG. 2 by differing shades.

Amplification with a forward universal primer and a single reverse target specific primer (primer pair 011) can recall a subpool 012 of oligonucleotide from the second pool of oligonucleotides 012. The subpool 012 produced using primer pair 011 and the second pool of synthetic oligonucleotides from 010 can comprise a common forward universal binding site, a common reverse target specific primer binding site, and content corresponding to the reverse target specific primer binding site.

Amplification with a single forward universal primer and four distinct reverse target specific primers (primer pairs 013) can be used to recall a subpool of oligonucleotides 014 with four distinct subsets of sequences from the second pool of oligonucleotides 010. Each one of the four subsets in subpool 014 can comprise a common universal primer binding site, a common reverse target specific primer binding site, and content corresponding to the reverse target specific primer binding site.

Amplification with a single forward universal primer and two distinct reverse target specific primers (primer pairs 015) can be used to recall a subpool of oligonucleotides 016 with two distinct subsets of sequences from the second pool of oligonucleotides 010. Each one of the two subsets in subpool 016 can comprise a common universal primer binding site, a common reverse target specific primer binding site, and content corresponding to the reverse target specific primer binding site.

In some embodiments, the content portion of the second pool of oligonucleotides 010 can be destined for use in target enrichment of massively parallel DNA sequencing libraries as hybridization capture probes. In some embodiments, the content portion of the second pool of synthetic oligonucleotides 010 can be destined for use in target enrichment of massively parallel DNA sequencing libraries as PCR primers.

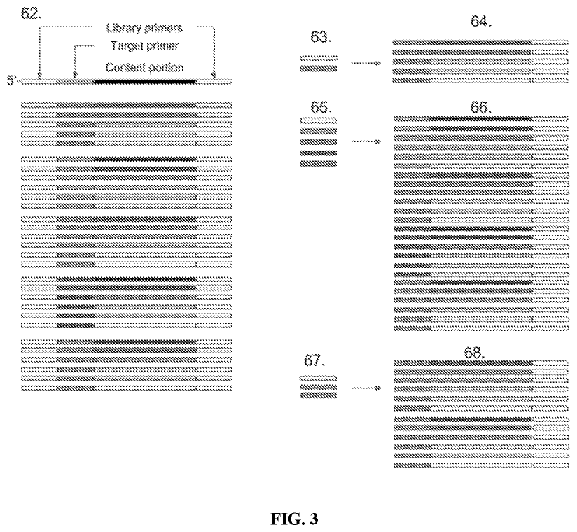

FIG. 3 depicts an exemplary construction of a third exemplary oligonucleotide pool 062, for which a subpool can be selectively amplified. Each molecule in the third exemplary pool of synthetic oligonucleotides can comprise in 5′ to 3′ order (left to right), a forward library primer sequence common to all molecules in the pool, a forward target specific primer binding site, a content portion, and a reverse library primer binding site common to all molecules in the pool. Different pairs of target specific amplification primers in the second oligonucleotide pool 062 are depicted in FIG. 3 with different shades. Different content sharing a same target specific primer pair is indicated in FIG. 3 by differing shades.

Amplification with a forward target specific primer and a single reverse universal primer (primer pair 063) can recall a subpool 064 of oligonucleotide from the third pool of oligonucleotides 022. The subpool 064 produced using primer pair 063 and the second pool of synthetic oligonucleotides from 062 can comprise a common forward target specific primer binding site, a common reverse universal primer binding site, and content corresponding to the forward target specific primer binding site.

Amplification with four distinct forward target specific primers and a single reverse universal primer (primer pairs 065) can be used to recall a subpool of oligonucleotides 066 with four distinct subsets of sequences from the third pool of oligonucleotides 062. Each one of the four subsets in subpool 066 can comprise a common forward target specific primer binding site, a common reverse universal primer binding site, and content corresponding to the forward target specific primer binding site.

Amplification with two distinct forward target specific primers and a single reverse universal primer (primer pairs 067) can be used to recall a subpool of oligonucleotides 068 with two distinct subsets of sequences from the third pool of oligonucleotides 062. Each one of the two subsets in subpool 068 can comprise a common forward target specific primer binding site, a common reverse universal primer binding site, and content corresponding to the forward target specific primer binding site.

In some embodiments, the content portion of the third pool of oligonucleotides 062 can be destined for use in target enrichment of massively parallel DNA sequencing libraries as hybridization capture probes. In some embodiments, the content portion of the third pool of synthetic oligonucleotides 062 can be destined for use in target enrichment of massively parallel DNA sequencing libraries as PCR primers.

In some embodiments, a forward universal primer sequence and a reverse universal primer binding site can be identical between every member of a pool of oligonucleotides as described herein. In some embodiments, a forward universal primer sequence and a reverse universal primer binding site can be identical between a plurality of distinct members of a pool of oligonucleotides having distinct forward target specific primer sequence and reverse target specific primer binding sites. A plurality of distinct members of a pool of oligonucleotides with distinct content, forward target specific primer sequence, and reverse target specific primer binding sites can be amplified by e.g. PCR from a pool of synthetic oligonucleotides using a single forward library primer and a single reverse library primer.

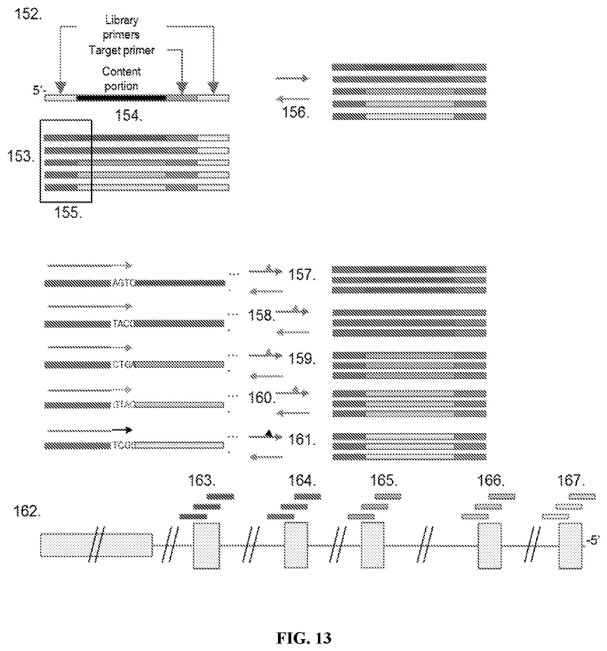

While some embodiments depict amplification using primers common to all members of a subpool, subsets of a subpool can be recalled using specific variations of primers as described herein. FIG. 13 depicts a method of recalling smaller subsets of a subpool from the second exemplary pool 010 depicted in FIG. 2. As depicted in FIG. 13, each oligonucleotide in the subpool 152 can comprise in 5′ to 3′ order (left to right), a forward library primer sequence common to all molecules in the pool, a content portion, a reverse target specific primer binding site, and a reverse library primer binding site common to all molecules in the pool. A subset 153 of the subpool 152 can comprise the same forward library primer sequence and the same reverse target specific primer binding site. Amplification with a primer pair 156 containing a forward library primer and a reverse content specific primer complementary to the forward library primer sequence and the reverse target specific primer binding site can produce a subset 153 with content spanning desired loci as described above. However, it is possible to differentiate the subset 153 into additional subsets of subset 153 using specific primers. In some cases, a modified library primer can be used. A modified library primer can be complimentary to a forward library primer sequence, and can include a 5′ portion of a content region. Accordingly, amplification with a primer pair comprising a reverse content specific primer and modified library primer 157, 158, 159, 160, or 161 can produce subsets of subset 153 that can correspond to individual loci. As illustrated in FIG. 13, amplification using modified library primer 157 can produce primers with content spanning locus 163, amplification using modified library primer 158 can produce primers with content spanning locus 164, amplification using modified library primer 159 can produce primers with content spanning locus 165, amplification using modified library primer 160 can produce primers with content spanning locus 166, and amplification using modified library primer 161 can produce primers with content spanning locus 167.

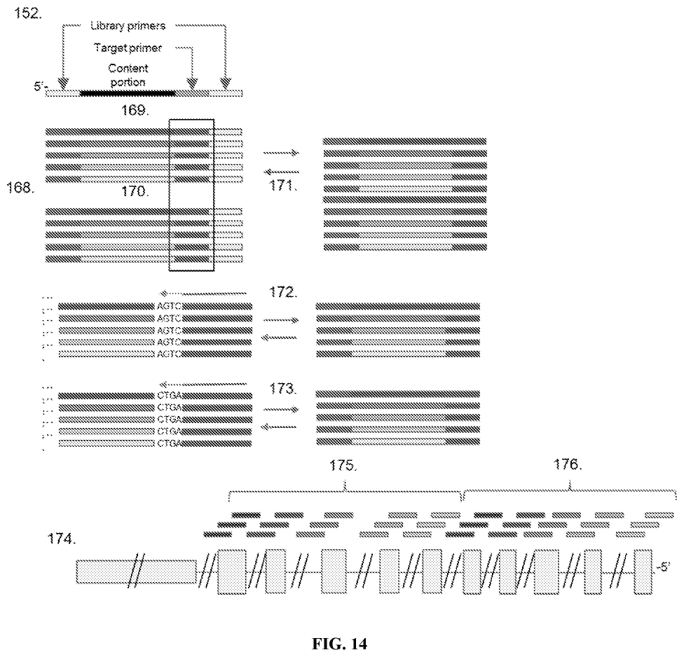

In some embodiments, amplification with a modified target specific primer can be used to recall a subset of a subset. FIG. 14 depicts another method of recalling smaller subsets of a subpool from the second exemplary pool 010 depicted in FIG. 2. As depicted in FIG. 14, each oligonucleotide in the subpool 152 can comprise in 5′ to 3′ order (left to right), a forward library primer sequence common to all molecules in the pool, a content portion, a reverse target specific primer binding site, and a reverse library primer binding site common to all molecules in the pool. A subset 168 of the subpool 152 can comprise the same forward library primer sequence and the same reverse target specific primer binding site, and can contain subsets 169 and 170 with distinct content regions. Amplification with a primer pair 171 containing a forward library primer and a reverse content specific primer complementary to the forward library primer sequence and the reverse target specific primer binding site can produce subset 168 containing both subset 169 and 170. However, it is possible to differentiate between subset 169 and 170 using specific primers. In some cases, a modified content specific primer can be used. A content library primer can be complimentary to a reverse target specific primer binding site, and can include a 3′ portion of a content region. Accordingly, amplification with a primer pair comprising a reverse content specific primer and modified library primer 172 or 173 can produce subsets of subsets 169 or 170, respectively. As illustrated in FIG. 14, amplification using modified content specific primer 172 can produce primers with content spanning locus 175, and amplification using modified content specific primer 173 can produce primers with content spanning locus 176.

In some embodiments, a forward target specific primer sequence and a reverse target specific primer binding site can be present on the same member of a pool of synthetic oligonucleotides and can be referred to as a target specific primer pair. A plurality of distinct target specific primer pairs may be present in a pool of synthetic oligonucleotides. In some cases, there may be from about 10 to about 100, distinct target specific primer pairs, from about 100 to about 1000 distinct target specific primer pairs, or from about 1000 to about 10,000 distinct target specific primer pairs. In some embodiments, a target specific primer pair can flank from about 1 to about 10, from about 10 to about 100, from about 100 to about 1000, or from about 1,000 to about and 10,000 distinct content sequences, and can therefore be used to amplify as many distinct content sequences when used as primers in a PCR from a pool of oligonucleotides.

Processing of Oligonucleotide Subpools

Disclosed herein are methods of processing oligonucleotides. An oligonucleotide can be a member of a pool or a subpool. In some cases, a subpool can be prepared as described herein.

A subpool of oligonucleotides can be processed for use in high throughput sequencing. Processing can be used to prepare hybridization capture probes or PCR primers for use in high throughput sequencing from a subpool of oligonucleotide primers.

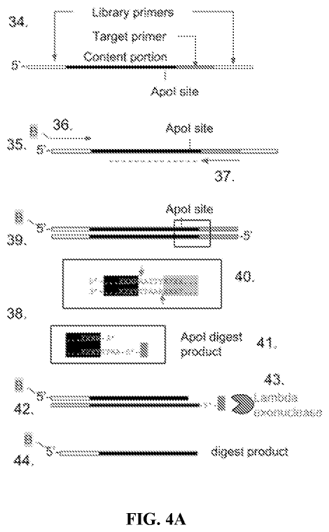

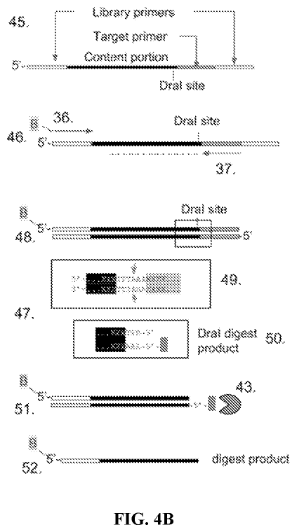

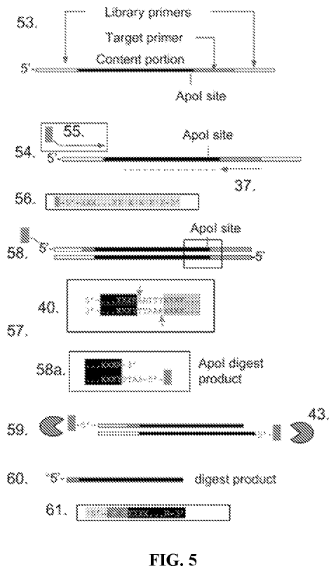

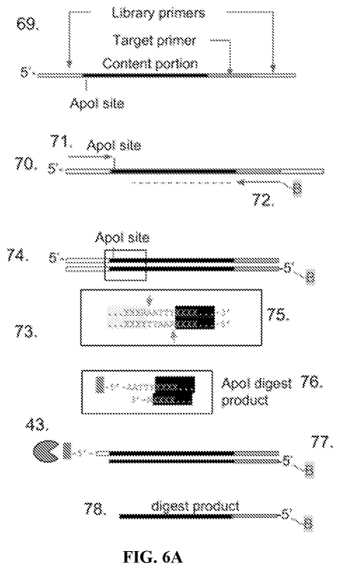

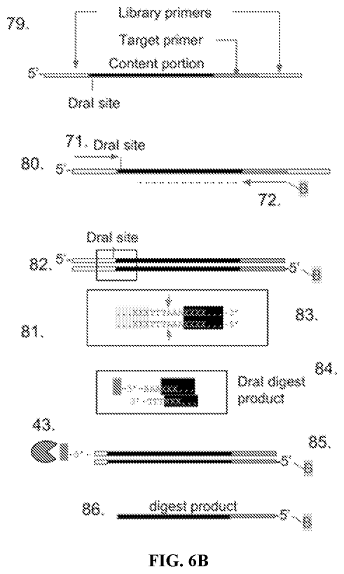

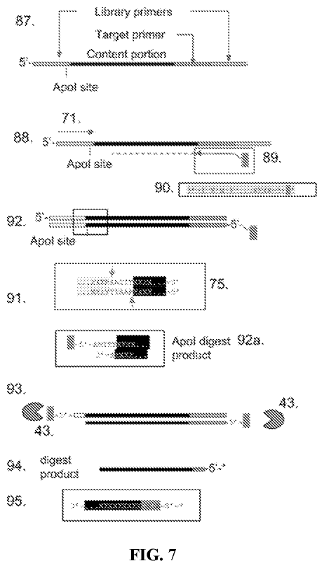

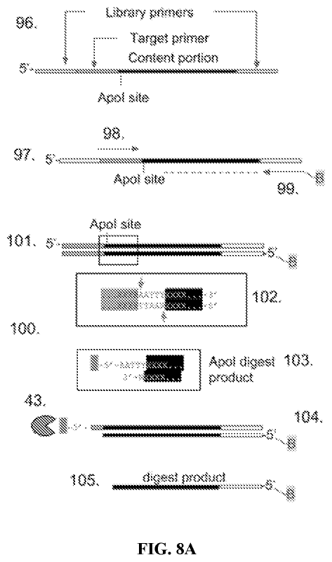

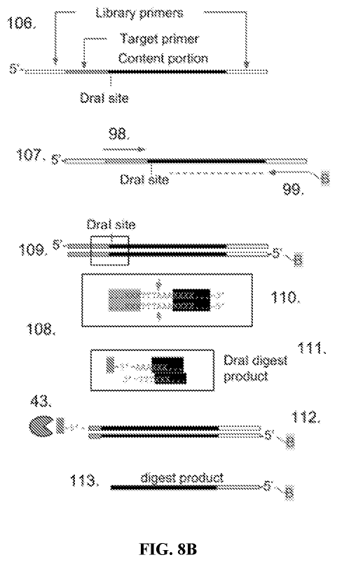

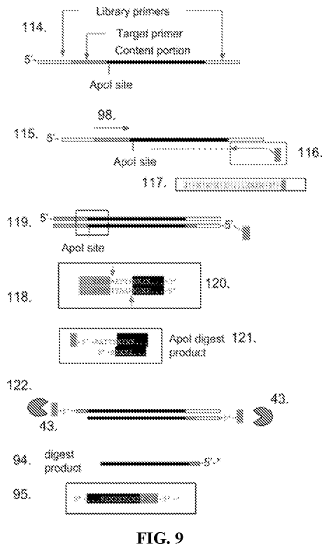

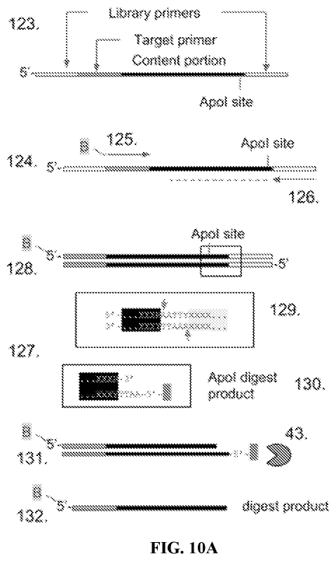

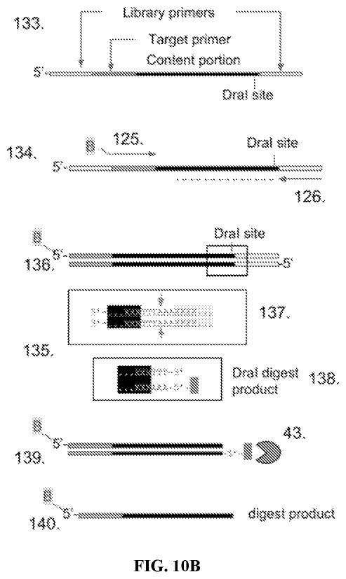

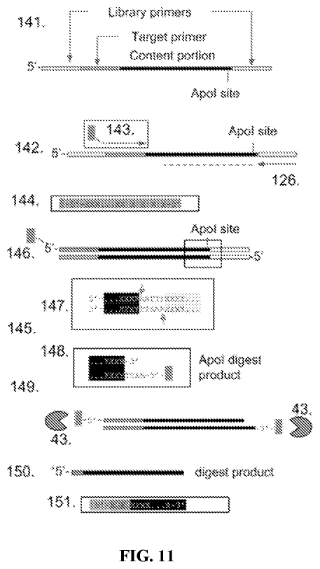

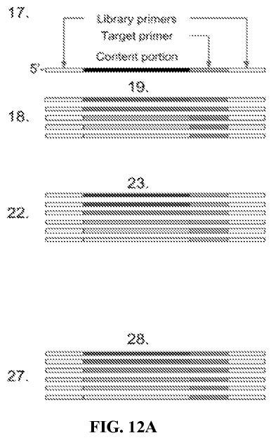

A single stranded oligonucleotide can be prepared using enzymatic transformation of a duplex as described below. FIGS. 4A, 4B, and 5 depict methods of preparing a single stranded sense probe from oligonucleotides from the second exemplary subpool 010, while FIGS. 6A, 6B and 7 depict methods of preparing a single stranded antisense probe from oligonucleotides from the second exemplary subpool 010. FIGS. 8A, 8B, and 9 depict methods of preparing a single stranded antisense probe from oligonucleotides from the third exemplary subpool 062, while FIGS. 10A, 10B and 11 depict methods of preparing a single stranded sense probe from oligonucleotides from the third exemplary subpool 062.

A hybridization capture probe can be labeled with a recognition element. While examples below depict a recognition element as biotin, a recognition element is not limited to biotin. In some cases, a recognition element can be added to a hybridization probe by use of biotinylated nucleotide triphosphates, use of activating groups on primers or digoxigenin labeled primers, or other means of labeling the PCR product for specific purification.

FIG. 4A depicts preparation of biotinylated hybridization probes 044 from a subset 034 of oligonucleotides present in the second exemplary pool 010 depicted in FIG. 2. As depicted in FIG. 4A, each oligonucleotide in the subset 034 can comprise in 5′ to 3′ order (left to right), a forward library primer sequence common to all molecules in the pool, a content portion, a recognition sequence for an ApoI restriction endonuclease, reverse target specific primer binding site, and a reverse library primer binding site common to all molecules in the pool. PCR amplification can be performed using a biotinylated forward library primer 036 and an unmodified reverse target specific primer 037 to produce double stranded product 039. The double stranded product 039 can comprise an ApoI recognition site 040. As depicted in FIG. 4A, ‘X’ can represent any base, ‘R’ can represent an ‘A’ or a ‘G’, and Y can represent a ‘C’ or a ‘T.’ Digestion with an ApoI restriction enzyme can produce a biotinylated duplex 042 with a 3′ “sticky end,” where a forward strand can terminate in an ‘R’ residue and a non-biotinylated reverse strand can carry a 5′ phosphate after ApoI digestion, as indicated with a light shaded rectangle at the 5′ end of the digest site. Digestion with an exonuclease 043 (e.g. lambda exonuclease) with 5′ to 3′ exonuclease activity can produce a biotinylated single stranded sense oligonucleotide 044, which can be used as a hybridization capture probe.

FIG. 4B depicts an additional method for preparation of biotinylated hybridization probes 052 from a subset 045 of oligonucleotides present in the second exemplary pool 010 depicted in FIG. 2. As depicted in FIG. 4B, each oligonucleotide in the subset 045 can comprise in 5′ to 3′ order (left to right), a forward library primer sequence common to all molecules in the pool, a content portion, a recognition sequence for a DraI restriction endonuclease, reverse target specific primer binding site, and a reverse library primer binding site common to all molecules in the pool. PCR amplification can be performed using a biotinylated forward library primer 036 and an unmodified reverse target specific primer 037 to produce double stranded product 048. The double stranded product 048 can comprise a DraI recognition site 049. As depicted in FIG. 4B, ‘X’ can represent any base. Digestion with a DraI restriction enzyme can produce a biotinylated duplex 051 with a 3′ “blunt end,” where a biotinylated forward strand can terminate in exogenous “TTT” sequence and a non-biotinylated reverse strand can carry a 5′ phosphate after DraI digestion, as indicated with a light shaded rectangle at the 5′ end of the digest site. Digestion with an exonuclease 043 (e.g. lambda exonuclease) with 5′ to 3′ exonuclease activity can produce a biotinylated single stranded sense oligonucleotide 052, which can be used as a hybridization capture probe.

FIG. 5 depicts preparation of extendable target enrichment primers 060 from a subset 053 of oligonucleotides present in the second exemplary pool 010 depicted in FIG. 2. As depicted in FIG. 5, each oligonucleotide in the subset 053 can comprise in 5′ to 3′ order (left to right), a forward library primer sequence common to all molecules in the pool, a content portion, a recognition sequence for an ApoI restriction endonuclease, reverse target specific primer binding site, and a reverse library primer binding site common to all molecules in the pool. PCR amplification can be performed using a modified forward library primer 055 and an unmodified reverse target specific primer 037 to produce double stranded product 058. The modified forward library primer 055 can be 5′ phosphorylated, indicated by the light shaded rectangle, and can have stability enhancing modifications near its 3′ end. A stability enhancing modification can include a phosphorothioate linkage between bases, as depicted by “*” in FIG. 5. The double stranded product 058 can comprise an ApoI recognition site 040. Digestion with an ApoI restriction enzyme can produce a duplex 059 with a 3′ “sticky end,” where each strand can terminate in a 5′ phosphate after ApoI digestion, as indicated with light shaded rectangles. Digestion with an exonuclease 043 (e.g. lambda exonuclease) with 5′ to 3′ exonuclease activity can digest both strands of duplex 059. However, the presence of phosphorothioate linkages on the 5′ strand can protect the 5′ strand from complete digestion. Accordingly, digestion of duplex 059 with an exonuclease can produce a single stranded sense oligonucleotide 061, which can be used as an extendable target enrichment primer or reagent.

FIG. 6A depicts preparation of antisense biotinylated hybridization probes 078 from a subset 069 of oligonucleotides present in the second exemplary pool 010 depicted in FIG. 2. As depicted in FIG. 6A, each oligonucleotide in the subset 069 can comprise in 5′ to 3′ order (left to right), a forward library primer sequence common to all molecules in the pool, a recognition sequence for the ApoI restriction endonuclease, a content portion, reverse target specific primer binding site, and a reverse library primer binding site common to all molecules in the pool. PCR amplification can be performed using an unmodified forward library primer 071 and a biotinylated reverse target specific primer 072 to produce double stranded product 074. The double stranded product 074 can comprise an ApoI recognition site 075. As depicted in FIG. 6A, ‘X’ can represent any base, ‘R’ can represent an ‘A’ or a ‘G’, and Y can represent a ‘C’ or a ‘T.’ Digestion with an ApoI restriction enzyme can produce a biotinylated duplex 077 with a 5′ “sticky end,” where a reverse strand can terminate in an ‘R’ residue and a non-biotinylated forward strand can carry a 5′ phosphate after ApoI digestion, as indicated with a light shaded rectangle. Digestion with an exonuclease 043 (e.g. lambda exonuclease) with 5′ to 3′ exonuclease activity can produce a biotinylated single stranded antisense oligonucleotide 078, which can be used as a hybridization capture probe.

FIG. 6B depicts an additional method for preparation of biotinylated antisense hybridization probes 086 from a subset 079 of oligonucleotides present in the second exemplary pool 010 depicted in FIG. 2. As depicted in FIG. 6B, each oligonucleotide in the subset 079 can comprise in 5′ to 3′ order (left to right), a forward library primer sequence common to all molecules in the pool, a recognition sequence for the DraI restriction endonuclease, a content portion, reverse target specific primer binding site, and a reverse library primer binding site common to all molecules in the pool. PCR amplification can be performed using an unmodified forward library primer 071 and a biotinylated reverse target specific primer 072 to produce double stranded product 082. The double stranded product 082 can comprise a DraI recognition site 083. As depicted in FIG. 6B, ‘X’ can represent any base. Digestion with a DraI restriction enzyme can produce a biotinylated duplex 085 with a 5′ “blunt end,” where a biotinylated reverse strand can terminate in exogenous “TTT” sequence and a forward strand can carry a 5′ phosphate after DraI digestion, as indicated with a light shaded rectangle. Digestion with an exonuclease 043 (e.g. lambda exonuclease) with 5′ to 3′ exonuclease activity can produce a biotinylated single stranded antisense oligonucleotide 086, which can be used as a hybridization capture probe.

FIG. 7 depicts preparation of extendable target enrichment primers 094 from a subset 087 of oligonucleotides present in the second exemplary pool 010 depicted in FIG. 2. As depicted in FIG. 7, each oligonucleotide in the subset 087 can comprise in 5′ to 3′ order (left to right), a forward library primer sequence common to all molecules in the pool, a recognition sequence for the ApoI restriction endonuclease, a content portion, reverse target specific primer binding site, and a reverse library primer binding site common to all molecules in the pool. PCR amplification can be performed using an unmodified forward library primer 071 and a modified reverse target specific primer 089 to produce double stranded product 092. The modified reverse target specific primer 090 can be 5′ phosphorylated, indicated by the light shaded rectangle, and can have stability enhancing modifications near its 3′ end. A stability enhancing modification can include a phosphorothioate linkage between bases, as depicted by “*” in FIG. 7. The double stranded product 092 can comprise an ApoI recognition site 075. Digestion with an ApoI restriction enzyme can produce a duplex 093 with a 5′ “sticky end,” where each strand can carry a 5′ phosphate after ApoI digestion, as indicated with a light shaded rectangle. Digestion with an exonuclease 043 (e.g. lambda exonuclease) with 5′ to 3′ exonuclease activity can digest both strands of duplex 093. However, the presence of phosphorothioate linkages on the 3′ strand can protect the 3′ strand from complete digestion. Accordingly, digestion of duplex 093 with an exonuclease can produce a single stranded antisense oligonucleotide 094, which can be used as an extendable target enrichment primer.

FIG. 8A depicts preparation of antisense biotinylated hybridization probes 105 from a subset 096 of oligonucleotides present in the third exemplary pool 062 depicted in FIG. 3. As depicted in FIG. 8A, each oligonucleotide in the subset 096 can comprise in 5′ to 3′ order (left to right), a forward library primer sequence common to all molecules in the pool, a forward target specific primer sequence, a recognition sequence for the ApoI restriction endonuclease, a content portion, and a reverse library primer binding site common to all molecules in the pool. PCR amplification can be performed using an unmodified forward target specific primer 098 and a biotinylated reverse library primer 099 to produce double stranded product 101. The double stranded product 101 can comprise an ApoI recognition site 102. As depicted in FIG. 8A, ‘X’ can represent any base, ‘It’ can represent an ‘A’ or a ‘G’, and Y can represent a ‘C’ or a ‘T.’ Digestion with an ApoI restriction enzyme can produce a biotinylated duplex 104 with a 5′ “sticky end,” where a reverse strand can terminate in an ‘R’ residue and a non-biotinylated forward strand can carry a 5′ phosphate after ApoI digestion, as indicated with a light shaded rectangle. Digestion with an exonuclease 043 (e.g. lambda exonuclease) with 5′ to 3′ exonuclease activity can produce a biotinylated single stranded antisense oligonucleotide 105, which can be used as a hybridization capture probe.

FIG. 8B depicts an additional method for preparation of biotinylated antisense hybridization probes 113 from a subset 106 of oligonucleotides present in the third exemplary pool 062 depicted in FIG. 3. As depicted in FIG. 8B, each oligonucleotide in the subset 106 can comprise in 5′ to 3′ order (left to right), a forward library primer sequence common to all molecules in the pool, a forward target specific primer sequence, a recognition sequence for the DraI restriction endonuclease, a content portion, and a reverse library primer binding site common to all molecules in the pool. PCR amplification can be performed using an unmodified forward target specific primer 098 and a biotinylated reverse library primer 099 to produce double stranded product 109. The double stranded product 109 can comprise a DraI recognition site 110. As depicted in FIG. 8B, ‘X’ can represent any base. Digestion with a DraI restriction enzyme can produce a biotinylated duplex 112 with a 5′ “blunt end,” where a biotinylated reverse strand can terminate in exogenous “TTT” sequence and a forward strand can carry a 5′ phosphate after DraI digestion, as indicated with a highlighted capital ‘P’. Digestion with an exonuclease 043 (e.g. lambda exonuclease) with 5′ to 3′ exonuclease activity can produce a biotinylated single stranded antisense oligonucleotide 113, which can be used as an antisense hybridization capture probe.

FIG. 9 depicts preparation of extendable target enrichment primers 094 from a subset 114 of oligonucleotides present in the third exemplary pool 062 depicted in FIG. 3. As depicted in FIG. 9, each oligonucleotide in the subset 114 can comprise in 5′ to 3′ order (left to right), a forward library primer sequence common to all molecules in the pool, a forward target specific primer sequence, a recognition sequence for the ApoI restriction endonuclease, a content portion, and a reverse library primer binding site common to all molecules in the pool. PCR amplification can be performed using an unmodified target specific primer 098 and a modified reverse library primer 116 to produce double stranded product 119. The modified reverse library primer 116 can be 5′ phosphorylated, indicated by the light shaded rectangle, and can have stability enhancing modifications near its 3′ end. A stability enhancing modification can include a phosphorothioate linkage between bases, as depicted by “*” in FIG. 9. The double stranded product 119 can comprise an ApoI recognition site 075. Digestion with an ApoI restriction enzyme can produce a duplex 122 with a 5′ “sticky end,” where each strand can carry a 5′ phosphate after ApoI digestion, as indicated with a light shaded rectangle. Digestion with an exonuclease 043 (e.g. lambda exonuclease) with 5′ to 3′ exonuclease activity can digest both strands of duplex 122. However, the presence of phosphorothioate linkages on the 3′ strand can protect the 3′ strand from complete digestion. Accordingly, digestion of duplex 122 with an exonuclease can produce a single stranded antisense oligonucleotide 094, which can be used as an extendable target enrichment primer.

FIG. 10A depicts preparation of biotinylated hybridization probes 132 from a subset 123 of oligonucleotides present in the third exemplary pool 062 depicted in FIG. 3. As depicted in FIG. 10A, each oligonucleotide in the subset 123 can comprise in 5′ to 3′ order (left to right), a forward library primer sequence common to all molecules in the pool, a forward target specific primer sequence, a content portion, a recognition sequence for the ApoI restriction endonuclease, and a reverse library primer binding site common to all molecules in the pool. PCR amplification can be performed using a biotinylated forward target specific primer 125 and an unmodified reverse library primer 126 to produce double stranded product 128. The double stranded product 128 can comprise an ApoI recognition site 129. As depicted in FIG. 10A, ‘X’ can represent any base, ‘It’ can represent an ‘A’ or a ‘G’, and Y can represent a ‘C’ or a ‘T.’ Digestion with an ApoI restriction enzyme can produce a biotinylated duplex 131 with a 3′ “sticky end,” where a forward strand can terminate in an ‘R’ residue and a non-biotinylated reverse strand can carry a 5′ phosphate after ApoI digestion, as indicated with a shaded rectangle. Digestion with an exonuclease 043 (e.g. lambda exonuclease) with 5′ to 3′ exonuclease activity can produce a biotinylated single stranded sense oligonucleotide 132, which can be used as a sense hybridization capture probe.

FIG. 10B depicts an additional method for preparation of biotinylated hybridization probes 140 from a subset 133 of oligonucleotides present in the third exemplary pool 062 depicted in FIG. 3. As depicted in FIG. 10B, each oligonucleotide in the subset 133 can comprise in 5′ to 3′ order (left to right), a forward library primer sequence common to all molecules in the pool, a forward target specific primer sequence, a content portion, a recognition sequence for the DraI restriction endonuclease, and a reverse library primer binding site common to all molecules in the pool. PCR amplification can be performed using a biotinylated forward target specific primer 125 and an unmodified reverse library primer 126 to produce double stranded product 136. The double stranded product 136 can comprise a DraI recognition site 137. As depicted in FIG. 10B, ‘X’ can represent any base. Digestion with a DraI restriction enzyme can produce a biotinylated duplex 139 with a 3′ “blunt end,” where a biotinylated forward strand can terminate in exogenous “TTT” sequence and a non-biotinylated reverse strand can carry a 5′ phosphate after DraI digestion, as indicated with a shaded rectangle. Digestion with an exonuclease 043 (e.g. lambda exonuclease) with 5′ to 3′ exonuclease activity can produce a biotinylated single stranded sense oligonucleotide 140, which can be used as a sense hybridization capture probe.

FIG. 11 depicts preparation of extendable target enrichment primers 150 from a subset 141 of oligonucleotides present in the third exemplary pool 062 depicted in FIG. 3. As depicted in FIG. 11, each oligonucleotide in the subset 141 can comprise in 5′ to 3′ order (left to right), a forward library primer sequence common to all molecules in the pool, a forward target specific primer sequence, a content portion, a recognition sequence for the ApoI restriction endonuclease, and a reverse library primer binding site common to all molecules in the pool. PCR amplification can be performed using a modified forward target specific primer 143 and an unmodified reverse library primer 126 to produce double stranded product 146. The modified forward target specific primer 143 can be 5′ phosphorylated, indicated by the shaded rectangle and can have stability enhancing modifications near its 3′ end. A stability enhancing modification can include a phosphorothioate linkage between bases, as depicted by “*” in FIG. 11. The double stranded product 146 can comprise an ApoI recognition site 147. Digestion with an ApoI restriction enzyme can produce a duplex 149 with a 3′ “sticky end,” where each strand can terminate in a 5′ phosphate after ApoI digestion, as indicated with a shaded rectangle. Digestion with an exonuclease 043 (e.g. lambda exonuclease) with 5′ to 3′ exonuclease activity can digest both strands of duplex 149. However, the presence of phosphorothioate linkages on the 5′ strand can protect the 5′ strand from complete digestion. Accordingly, digestion of duplex 149 with an exonuclease can produce a single stranded sense oligonucleotide 150, which can be used as an extendable target enrichment primer.

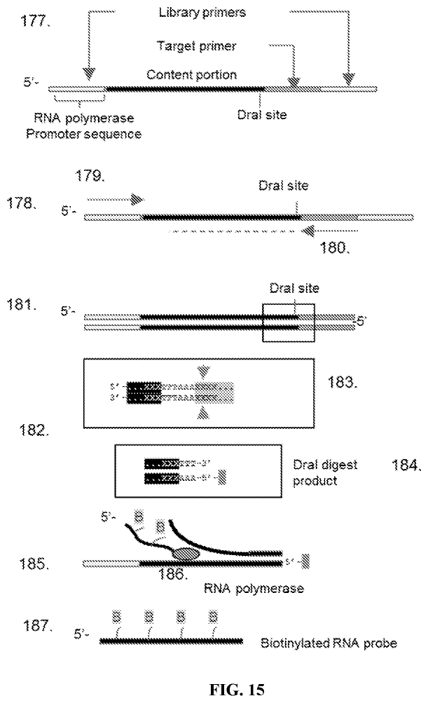

Enzymatic processing of subpools can be used to prepare RNA probes using similar methods as described above. FIG. 15 depicts an exemplary method of preparing RNA probes from the second exemplary pool 010 of FIG. 2. Each oligonucleotide in the subset 177 can comprise in 5′ to 3′ order (left to right), a forward library primer sequence common to all molecules in the second exemplary pool 010, a content portion, a site specific endonuclease (in this example, DraI), a reverse target specific primer binding site, and a reverse library primer binding site common to all molecules in the second exemplary pool 101. In some cases, a subpool 177 can comprise a promoter sequence for an RNA polymerase (e.g. T7 RNA polymerase) within the forward library primer sequence. PCR amplification can be performed using an forward library primer 179 and a reverse target specific primer 180 to produce double stranded product 181. The double stranded product 181 can comprise a DraI recognition site 183. As depicted in FIG. 15, ‘X’ can represent any base. Digestion with a DraI restriction enzyme can produce a duplex 184 with a 3′ “blunt end,” where a forward strand can terminate in exogenous “TTT” sequence and a reverse strand can carry a 5′ phosphate after DraI digestion, as indicated with a light shaded rectangle. In vitro transcription can be performed using an RNA polymerase 186 and affinity labeled nucleotides, such as biotinylated nucleotides. The exemplary illustration of FIG. 15 depicts transcription initiating from a 3′ end of the RNA polymerase promoter sequence. In vitro transcription of the duplex 184 can produce affinity labeled RNA probes 187.

While specific endonucleases have been highlighted above, it is understood that other endonucleases can be substituted. Substitution of other restriction enzymes can require mutation of a corresponding restriction endonuclease recognition site within a pool or subpool. In some cases, a restriction enzyme can include: Aar I, BanII, BseGI, BspPI, CfrI, EcoNI, Hsp92II, NlaIV, RsaI, TaiI, AasI, BbsI, BseJI, BspTI, ClaI, EcoO109I, I-PpoI, NmuCI, RsrII, TaqaI, AatII, BbuI, BseLI, BsrBI, CpoI, EcoRI, KasI, NotI, SacI, TaqI, Acc65I, BbvCI, BseMI, BsrDI, Csp45I, EcoRV, Kpn2I, NruI, SacII, TasI, AccB7I, BbvI, BseMII, BsrFI, Csp6I, EheI, KpnI, NsbI, SalI, TatI, AccI, BceAI, BseNI, BsrGI, CspI, Esp3I, KspAI, NsiI, SapI, TauI, AccIII, BcgI, BseRI, BsrI, DdeI, FauI, LweI, NspI, SatI, TfiI, AciI, BciVI, BseSI, BsrSI, DpnI, Fnu4HI, MbiI, OliI, Sau3AI, TliIAclI, BclI, BseXI, BssHII, DpnII, FokI, MboI, PacI, Sau96I, TrulI, AdeI, BcnI, BseYI, BssKI, DraI, FseI, MboII, PaeI, SbfI, Tru9I, AfeI, BcuI, BsgI, BssSI, DraIII, FspAI, MfeI, PaeR7I, ScaI, TseI, AflII, BfaI, Bsh1236I, Bst1107I, DrdI, FspI, MlsI, PagI, SchI, Tsp45I, AflIII, BfiI, Bsh12851, Bst98I, EaeI, GsuI, MluI, PauI, ScrFI, Tsp509I, AgeI, BfmI, BshNI, BstAPI, EagI, HaeII, MlyI, PciI, SdaI, TspRI, AhdI, BfrBI, BshTI, BstBI, Eam1104I, HaeIII, MmeI, PdiI, SduI, Tth111I, AleI, BfuAI, BsiEI, BstEII, Eam1105I, HgaI, MnlI, PdmI, SexAI, NaeI, AloI, BfuCI, BsiHKAI, BstF5I, EarI, HhaI, Mph1103I, Pfl23II, SfaNI, AluI, BfuI, BsiWI, BstNI, EciI, HinlI, MscI, PflFI, SfcI, Van91I, Alw21I, BglI, BslI, BstOI, Ec1136II, Hin4I, MseI, PflMI, SfiI, VspI, Alw26I, BglII, BsmAI, BstUI, Ec1HKI, Hin6I, MslI, PfoI, SfoI, XagI, Alw44I, BlpI, BsmBI, BstXI, Eco105I, HincII, MspAlI, PleI, SgfI, XapI, AlwI, Bme1390I, BsmFI, BstYI, Eco130I, HindIII, MspI, PmeI, SgrAI, XbaI, AlwNI, BoxI, BsmI, BstZI, Eco147I, HinfI, MssI, PmiI, SinI, XceI, ApaI, BpiI, BsoBI, Bsu15I, Eco24I, HinPlI, MunI, PpiI, SmaI, XcmI, ApaLI, BplI, Bsp119I, Bsu36I, Eco31I, HpaI, Mva1269I, PpuMI, SmiI, XhoI, ApoI, Bpu10I, Bsp120I, BsuRI, Eco32I, HpaII, MvaI, PshAI, SmlI, XhoII, AscI, Bpu1102I, Bsp1286I, BtgI, Eco47I, HphI, MwoI, PsiI, SmuI, XmaI, AseI, BsaAI, Bsp1407I, BtsI, Eco47III, Hpy188I, NaeI, Psp1406I, SnaBI, XmaJI, AsiSI, BsaBI, Bsp143I, BveI, Eco52I, Hpy188III, NarI, Psp5II, SpeI, XmiI, AvaI, BsaHI, Bsp143II, Cac8I, Eco57I, Hpy8I, NciI, PspGI, SphI, XmnI, AvaII, BsaI, Bsp68I, CaiI, Eco57MI, Hpy99I, NcoI, PspOMI, SspI, AvrII, BsaJI, BspDI, CfoI, Eco72I, HpyCH4III, NdeI, PstI, StuI, BaeI, BsaMI, BspEI, Cfr10I, Eco81I, HpyCH4IV, NdeII, PsuI, StyD4I, BalI, BsaWI, BspHI, Cfr13I, Eco88I, HpyCH4V, NgoMIV, PsyI, StyI, BamHI, BsaXI, BspLI, Cfr42I, Eco91I, HpyF10VI, NheI, PvuI, SwaI, BanI, BseDI, BspMI, Cfr9I, EcoICRI, Hsp92I, NlaIII, PvuII, TaaI, or derivatives thereof. Further, alternative means of site specific endonucleolytic cleavage can be employed to remove primer regions from content, for example custom sgRNA directed cleavage by Cas9, or zinc finger endonuclease, or TALEN.

Use of Hybridization Capture Probes

A hybridization capture probe produced by any of the methods described herein can be used for high-throughput sequencing. In some embodiments, the content portion of a plurality of constructs within either the first, second, or third pools of synthetic oligonucleotides corresponds to fragments of human genomic content. In some embodiments, the genomic content of the first, second, or third pools of synthetic oligonucleotides is from a species other than human. In some embodiments, the content is generated to be used as a hybridization capture probe for target enrichment. In some embodiments, the content is used as hybridization capture probes for target enrichment of NGS libraries for targeted sequencing.

A pool or library of oligonucleotides can comprise oligonucleotides with content regions that can comprise a portion of a gene of interest. In some cases, a pool can contain a portion of a gene selected from the group consisting of: A2M, ABL1, ADCY5, AGPAT2, AGTR1, AIFM1, AKT1, APEX1, APOC3, APOE, APP, APTX, AR, ARHGAP1, ARNTL, ATF2, ATM, ATP5O, ATR, BAK1, BARD1, BAX, BCL2, BDNF, BLM, BMI1, BRCA1, BRCA2, BSCL2, BUB1B, BUB3, C1QA, CACNA1A, CAT, CCNA2, CDC42, CDK1, CDK7, CDKN1A, CDKN1B, CDKN2A, CDKN2B, CEBPA, CEBPB, CETP, CHEK2, CISD2, CLOCK, CLU, CNR1, COQ7, CREB1, CREBBP, CSNK1E, CTF1, CTGF, CTNNB1, DBN1, DDIT3, DGAT1, DLL3, E2F1, EEF1A1, EEF1E1, EEF2, EFEMP1, EGF, EGFR, EGR1, EIF5A2, ELN, EMD, EP300, EPOR, EPS8, ERBB2, ERCC1, ERCC2, ERCC3, ERCC4, ERCC5, ERCC6, ERCC8, ESR1, FAS, FEN1, FGF21, FGF23, FGFR1, FGFR2, FGFR3, FLT1, FOS, FOXM1, FOXO1, FOXO3, FOXO4, GCLC, GCLM, GDF11, GH1, GHR, GHRH, GHRHR, GPX1, GPX4, GRB2, GRN, GSK3A, GSK3B, GSR, GSS, GSTA4, GSTP1, GTF2H2, H2AFX, HBP1, HDAC1, HDAC2, HDAC3, HELLS, HESX1, HIC1, HIF1A, HMGB1, HMGB2, HNF1A, HOXB7, HOXC4, HRAS, HSF1, HSP90AA1, HSPA1A, HSPA1B, HSPA8, HSPA9, HSPD1, HTRA2, HTT, IFNB1, IGF1, IGF1R, IGF2, IGFBP2, IGFBP3, IKBKB, IL2, IL2RG, IL6, IL7, IL7R, INS, INSR, IRS1, IRS2, JAK1, JAK2, JAK3, JUN, JUND, KCNA3, KIT, KL, KRAS, LEP, LEPR, LMNA, LMNB1, LRP2, MAP3K5, MAPK14, MAPK3, MAPK8, MAPK9, MAPT, MAX, MDM2, MED1, MET, MIF, MLH1, MSRA, MT-CO1, MT1E, MTOR, MXD1, MXI1, MYC, NBN, NCOR1, NCOR2, NFE2L1, NFE2L2, NFKB1, NFKB2, NFKBIA, NGF, NGFR, NOG, NOTCH1, NR3C1, NRG1, NUDT1, PAPPA, PARP1, PCK1, PCMT1, PCNA, PDGFB, PDGFRA, PDGFRB, PDPK1, PEX5, PIK3CA, PIK3CB, PIK3R1, PIN1, PLAU, PLCG2, PMCH, PML, POLA1, POLB, POLD1, POLG, PON1, POU1F1, PPARA, PPARG, PPARGC1A, PPM1D, PPP1CA, PRDX1, PRKCA, PRKCD, PRKDC, PROP1, PSEN1, PTEN, PTGS2, PTK2, PTK2B, PTPN1, PTPN11, PYCR1, RAD51, RAD52, RAE1, RB1, RECQL4, RELA, RET, RGN, RICTOR, RPA1, S100B, SDHC, SERPINE1, SHC1, SIN3A, SIRT1, SIRT3, SIRT6, SIRT7, SLC13A1, SNCG, SOCS2, SOD1, SOD2, SP1, SPRTN, SQSTM1, SRCSST, SSTR3, STAT3, STAT5A, STAT5B, STK11, STUB1, SUMO1, SUN1, TAF1, TBP, TCF3, TERC, TERF1, TERF2, TERT, TFAP2A, TFDP1, TGFB1, TNF, TOP1, TOP2A, TOP2B, TOP3B, TP53, TP53BP1, TP63, TP73, TPP2, TRAP1, TRPV1, TXN, UBB, UBE2I. UCHL1, UCP1, UCP2, UCP3, VCP, VEGFA, WRN, XPA, XRCC5, XRCC6, YWHAZ, and ZMPSTE24.

A pool of primers can be constructed with content regions spanning a gene or portion thereof as described herein. In some cases, a pool or library can be present on a microarray or chip. In some cases, a pool or library can be present in solution. In some cases, a database can be constructed with sequences of genes covered by a pool of oligonucleotides. A system storing a database in memory can be used to access sequences of genes for constructing oligonucleotides. A system can comprise an algorithm for generating a target specific primer binding site that can correspond to a given gene or portion thereof. An operator can consult a system to prepare an oligonucleotide pool with genes of interest and corresponding target specific primer binding regions. In some cases, a system can comprise software for designing such oligonucleotides. Further, a database of prepared oligonucleotides can be constructed storing all oligonucleotides and other identifying information. For example, the database may store a list of genes, gene sequences, primer sequence, primer locations, oligonucleotide sequences, oligonucleotide pool storage locations, and the like.

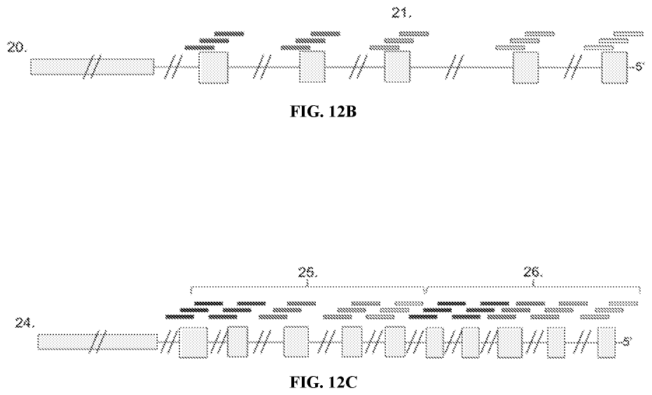

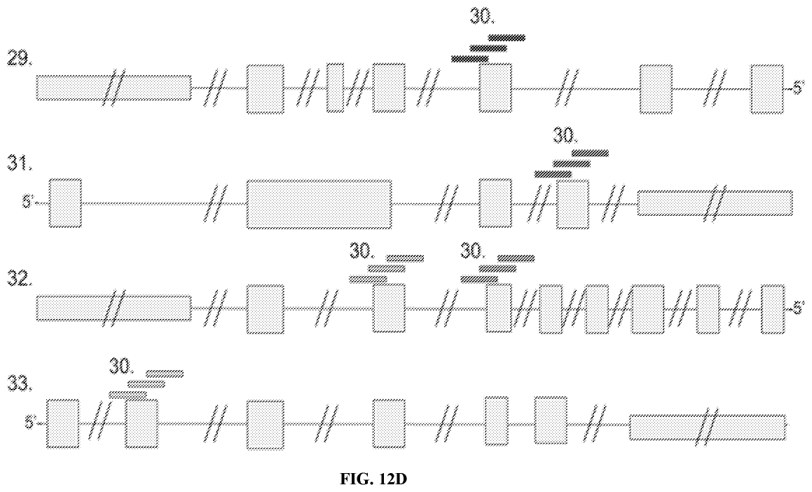

FIG. 12A depicts an overview of the use of a subpool of oligonucleotides isolated from the second exemplary pool of oligonucleotides depicted in FIG. 2 as target enrichment or sequencing primers. As described above, each molecule in the second exemplary pool 010 of oligonucleotides can comprise in 5′ to 3′ order (left to right), a forward library primer sequence common to all molecules in the pool, a content portion, a reverse target specific primer binding site, and a reverse library primer binding site common to all molecules in the pool.

FIGS. 12B-12D depict cartoon models of loci in a human genome. The entire gene region in FIGS. 12B-12D are indicated by horizontal lines, and exons are represented by boxes. For purposes of illustration, intron sizes are not to scale in FIGS. 12B-12D; nor are 3′ UTRs, which are indicated by pairs of slashes in FIGS. 12B-12D.

In some embodiments, a first subset of content can comprise fragments of genomic content that overlaps with protein coding exons of a human gene. FIG. 12B depicts a first set hybridization probes 021 prepared from a first subset 018 of a subpool recalled from a second exemplary pool 010. The first subset 018 of oligonucleotides in the second pool 010 of oligonucleotides can share the same reverse target specific primer binding site. Each member of the first subset 018 can have a different content, which is indicated by different shades. As illustrated in FIG. 12B, a first set hybridization probes 021 prepared from the first subset 018 can be used to sequence, for example, all exons of a locus without annealing to other regions of a locus.

In some embodiments, content can comprise fragments of genomic content, where more than one subset of content may be required to cover all coding exons of a gene. FIG. 12C depicts two sets hybridization probes 025 and 026 prepared from a second subset 022 of a subpool recalled from a second exemplary pool 010. The second subset 022 of oligonucleotides present in the second pool of oligonucleotides 010 can share the same reverse target specific primer binding site. Each member of the second subset 022 can have a different content, which is indicated by different shades. As illustrated in FIG. 12C, a set hybridization probes 025 prepared from the second subset 022 can be used to sequence, for example, a first half of coding exons in a locus, and can be recalled using a single primer pair. Similarly, a set hybridization probes 026 prepared from the second subset 022 can be used to sequence, for example, a second half of coding exons in a locus, and can be recalled using a single primer pair. Accordingly, a single forward library primer and two reverse target specific primers can prepare two subsets 025 and 026 of hybridization probes that can cover all coding exons of an exemplary locus.

In some cases, hybridization probes 030 spanning multiple loci can be generated from a subset 027 of a subpool recalled from a second exemplary pool 010. In some cases, a subset of content can comprise fragments of human genomic content that cover discrete regions across a plurality of distinct genes, for example all known oncogenic mutations in the RAS-MAP kinase pathway. As illustrated in FIG. 12D, the subset 027 of oligonucleotides present in the second pool of oligonucleotides 010 can share the same reverse target specific primer binding site. Each member of the second subset 027 can have a different content, which is indicated by different shades. As illustrated in FIG. 12D, a set hybridization probes 030 prepared from the subset 027 can be used to sequence coding exons across multiple loci, and can be recalled using a single primer pair.

In some instances, hybridization probes can be used to diagnose or monitor the progression of a disease or condition. In some cases, a disease can be an autoimmune disease such as acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal osteomyelitis (CRMO), Churg-Strauss syndrome, cicatricle pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophilic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosis, ligneous conjunctivitis, linear IgA disease (LAD), Lupus (SLE), Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricle pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuropathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease can be a cancer such as Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) cancer, Gastrointestinal Carcinoid Tumor, Gastrointestinal stromal tumor (GIST), Germ cell tumor: extracranial, extragonadal, or ovarian, Gestational trophoblastic tumor, Glioma of the brain stem, Glioma, Childhood Cerebral Astrocytoma, Glioma, Childhood Visual Pathway and Hypothalamic, Gastric carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Marcus Whittle, Deadly Disease, Macroglobulinemia, Waldenström, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, childhood, Salivary gland cancer, Sarcoma, Ewing family of tumors, Sarcoma, Kaposi, Sarcoma, soft tissue, Sarcoma, uterine, Sézary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma—see Skin cancer (nonmelanoma), Squamous neck cancer with occult primary, metastatic, Stomach cancer, Supratentorial primitive neuroectodermal tumor, childhood, T-Cell lymphoma, cutaneous—see Mycosis Fungoides and Sézary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, gestational, Unknown primary site, carcinoma of, adult, Unknown primary site, cancer of, childhood, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenström macroglobulinemia, Wilms tumor (kidney cancer), childhood.

In some cases, a disease can be inflammatory disease, infectious disease, cardiovascular disease and metabolic disease. Specific infectious diseases include, but is not limited to AIDS, anthrax, botulism, brucellosis, cancroid, chlamydial infection, cholera, coccidioidomycosis, cryptosporidiosis, cyclosporiasis, diphtheria, ehrlichiosis, arboviral encephalitis, enterohemorrhagic Escherichia coli, giardiasis, gonorrhea, dengue fever, Haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, Lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant Staphylococcus aureus, varicella, yellow fever, variant Creutzfeldt-Jakob disease (vCJD), Ebola hemorrhagic fever, Echinococcosis, Hendra virus infection, human monkeypox, influenza A, H5N1, lassa fever, Margurg hemorrhagic fever, Nipah virus, O'nyong fever, Rift valley fever, Venezuelan equine encephalitis and West Nile virus.

In some cases, a gene as disclosed herein can be implicated in a disease or condition as described herein. In some instances, a sample from a subject can be obtained to determine a sequence of at least one gene or portion thereof. A sample can be a biological sample. A biological sample may include a solid or liquid biopsy obtained from a source such as e.g. a cell, suspected or confirmed to harbor cancer. In some cases, a sample can be blood or any excretory liquid. Further non-limiting examples of the biological sample may include saliva, blood, serum, cerebrospinal fluid, semen, feces, plasma, urine, a suspension of cells, or a suspension of cells and viruses. A biological sample may contain whole cells, lysed cells, plasma, red blood cells, skin cells, proteins, nucleic acids (e.g. DNA, RNA, maternal DNA, maternal RNA), circulating nucleic acids (e.g. cell-free nucleic acids, cell-free DNA/cfDNA, cell-free RNA/cfRNA), circulating tumor DNA/ctDNA, cell-free fetal DNA/cffDNA).

Use of a hybridization probe can confirm a mutation of a gene or portion thereof known to cause a disease or condition as described herein. In some cases, a database storing a list of known mutations can be consulted based on the nature of the disease or condition. For example, a database of known breast cancer mutations can be consulted where a subject was previously diagnosed with breast cancer. Further, a subject's information can be stored on a database and accessed. In some instances, such information can include a subject's previous diagnosis, name age, height, weight, BMI, blood pressure, resting pulse, medical history, mental health status, sex, race, ethnicity, diet, or other risk factors such as smoking, drug or alcohol abuse, or potential drug incompatibilities.

A subject may be diagnosed with a disease or condition associated with a particular gene profile. For example, a subject may be diagnosed with one or more gene mutations spanning one or more genes indicative of a disease or condition. A physician, healthcare professional, laboratory professional, or the like may provide a gene profile. In some cases, a gene profile can be generated by sequencing nucleic acids obtained from a subject sample.

A hybridization probe can be used to monitor the progression of a treatment. For example, a subject previously diagnosed with a cancer who is receiving treatment (e.g. chemotherapy, gene therapy, CRISPR-CAS9) can provide biological samples over a period of time to monitor the effectiveness of treatment. In some cases, a subject can provide samples at least 1, 2, 3, 4, 5, 6, 7, 8, 9, 10, 11, 12, 13, 14, 15, 16, 17, 18, 19, 20, or 21 times a week. In some cases, a subject can provide samples at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some cases, a subject can provide samples at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, or 365 times per year.

### EXAMPLES

### Example 1—Amplification of a Synthetic Oligonucleotide Pool

A synthetic oligonucleotide pool is amplified with 30 cycles of PCR using a pair of universal amplification primers and a hi-fidelity DNA polymerase with 3′ to 5′ exonuclease (proofreading activity), e.g. Phusion DNA polymerase, in the appropriate reaction buffer supplemented with dNTP. Since every molecule in the pool of synthetic oligonucleotides 1) has regions which can be bound by the universal amplification primers and 2) these regions are at opposite termini of each construct, the entire pool of synthetic oligonucleotides and all content associated with each molecule can be amplified with the universal primer pair in a PCR reaction. Because chemical synthesis of long (>100 nt) oligonucleotides is imperfect and full-length synthesis products are a minority component of the overall synthesis, amplification of the pool by PCR is necessary to ensure that feedstock pool for subsequent reactions is solely comprised of full-length products. Amplified material is diluted in TE such that there are an average of 10,000 copies of each construct present per microliter.

### Example 2—Amplification of Subject-Specific Oligonucleotides from the Amplified Oligonucleotide Pool

Subject-specific oligonucleotides are amplified with 35 cycles of PCR from the dilution of the amplified synthetic oligonucleotide pool using a 5′ biotinylated universal primer and 1 to 16 different unmodified target specific reverse primers selected from the target database based on a subject's gene profile. The reaction is performed using Taq DNA polymerase in PCB supplemented with dNTP and using a concentration of biotinylated universal primer equal to the sum of all target specific reverse primers (200 nM to 3.2 uM final concentrations).

### Example 3—Cleavage of PCR Products

All constructs within the pool of synthetic oligonucleotides are designed such that each construct contains a DraI site between the content region of the oligo and the target specific primer binding region. In order to maximize probe yields, PCR products are not purified prior to the addition of DraI, as the enzyme is capable of complete cleavage in PCB.

### Example 4—Processing of Cut PCR Products into Single Stranded DNA Probes

Following cleavage with DraI, double-stranded probe sequences are converted into single stranded capture reagents by treatment with lambda exonuclease. Like DraI, Lambda exonuclease has 100% activity in PCB. Lambda exonuclease activity is stimulated by the presence of a 5′ phosphate and has greatly reduced activity against non-phosphorylated 5′ ends.

### Example 5—Purification of Biotinylated ssDNA Capture Probes

Following lambda exonuclease treatment, biotinylated capture probes are purified on SPRI beads (e.g. AMPure XP) using 2 to 3 volumes AMPure per reaction volume.

### Example 6—Construction of Shotgun Whole Genome Libraries

Sequencing libraries are constructed from at least 10 ng purified cfDNA extracted from plasma, urine, CSF, or other suitable bodily fluid. Briefly, purified cfDNA is end-repaired using a mixture of T4 DNA polymerase and T4 polynucleotide kinase and all four dNTP. Blunt and 5′ phosphorylated dsDNA is given a single dA tail using 3′ exonuclease deficient Klenow fragment of DNA polymerase I in a buffer containing dATP. Sequencing adapters containing molecular barcodes are ligated to 5′ phosphorylated and dA tailed dsDNA via sticky-end ligation using a single base T overhang on sequencing adapters in a reaction buffer containing T4 DNA ligase, ATP, and between 10 and 20% PEG-4000 to drive ligation to near completion. Ligated libraries are purified with SPRI beads using 1.5 volumes bead slurry per reaction volume. Sequencing ready libraries are amplified by 5 to 10 cycles of PCR using primers complementary to sequences present in sequencing adapters to yield 250 to 500 ng of amplified library. Amplified library is purified with SPRI beads using 1.5 volumes bead slurry per reaction volume.

### Example 7—Target Enrichment from Amplified Whole Genome Libraries

Amplified libraries are lyophilized with 5 μg of Cot-1 DNA and 1 nmol of sequencing adapter antisense blocking oligos. Dried DNA mixture is resuspended in hybridization buffer and blocking reagent and heated to 95° C. to denature DNA prior to hybridization. Denatured DNA is cooled to 65° C. and then mixed with 3 pmol of biotinylated ssDNA capture probes and held at this temperature for 4 hours. Probe bound library is recovered using 50-100 μL of Streptavidin coated magnetic beads and washed according to standard methods. Following washes, bound library is eluted from Streptavidin beads by incubating beads in 10 mM NaOH at 75° C. for 10 minutes. Library eluate from these beads is amplified by 10 to 20 cycles of PCR using primers complementary to sequences present in sequencing adapters to yield up to 250 ng of enriched library. Following PCR, enriched library is purified with SPRI beads using 1.5 volumes bead slurry per reaction volume. Singly enriched library is combined with 2.5 μg Cot-I DNA and 500 pmol of sequencing adapter antisense blocking oligos. A second round of capture is performed using 1.5 pmol of biotinylated ssDNA capture probes in the same manner as the first capture was performed. Following the second enrichment, eluted library is amplified with an additional 5 to 10 cycles of PCR using primers complementary to sequences present in sequencing adapters. Doubly enriched libraries are purified with SPRI beads using 1.5 volumes bead slurry per reaction volume. Library concentration is estimated by capillary gel electrophoresis or other similarly quantitative method and used to guide loading of a suitable sequencing instrument.

### Example 8—Obtaining a Subject Sample

A subject previously diagnosed with BRCAI-dependent breast cancer registers their information into a database. The subject provides their name, date of birth, height, weight, and medical history. A solid biopsy sample obtained from the subject is obtained and stored at −20° C. for further processing.

### Example 9—Consulting a Database to Access a Primer

A subject previously diagnosed with BRCAI-dependent breast cancer is admitted for monitoring. After a diagnosis of BRCAI-dependent breast cancer is input in a patient database, the location of sequence of primers sufficient to recall hybridization capture probes to sequence the BRCAI gene are displayed.

A laboratory technician accessing the database obtains a 5′ biotinylated universal primer and 3′ target specific primer to recall the hybridization capture probes. PCR amplification of the subject specific probes proceeds as described above in Example 2.

### Example 10—Isolation of Subject DNA

A lysis buffer containing 10 mM Tris, pH 7.5, 300 mM NaCl, 5 mM EDTA, 1% SDS and 30-50 μl 10 mg/ml proteinase K is added to portion of the biopsy sample. The suspension is incubated at 50° C. until tissue is digested. The suspension if centrifuged at 8,000 RPM at room temperature and the supernatant is collected.

Ethanol is added to the isolated supernatant and the resulting suspension is centrifuged. The pellet is collected and resuspended in TBS buffer. The resulting solution is subjected to sequencing using the subject-specific hybridization capture probes prepared in Example 9.

### Example 11—Monitoring the Progression of Breast Cancer

The subject previously diagnosed with breast cancer is biopsied once a month. A BRACI status is obtained by sequencing the BRCAI DNA from the biopsy sample after each visit as described in the Examples recited above.

As the subject continues to receive chemotherapy for treatment of BRCAI-dependent breast cancer, the presence of absence of known BRACI mutations implicated in breast cancer is followed over each subsequent visit. The elimination of aberrant DNA within the biopsy sample is indicative of a partial amelioration of the breast cancer in the subject.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

What is claimed is:

1. A method of preparing a subpool of subject-specific oligonucleotides from a first pool based on a gene profile from a subject, comprising:
a) obtaining the first pool, wherein the first pool comprises a plurality of oligonucleotides, and wherein each oligonucleotide comprises:
(i) a universal primer binding site, wherein each oligonucleotide of the plurality of oligonucleotides has the same universal primer binding site;
(ii) a content region comprising an overlap region to a portion of a gene, wherein the overlap region comprises at most 2 mismatches to the gene, wherein each oligonucleotide of the plurality of oligonucleotides has a different content region, and wherein a subset of oligonucleotides within the plurality of the oligonucleotides comprises a content region with at least a portion of a same gene; and
(iii) a target-specific primer binding site flanking content region;
b) amplifying exponentially from the first pool the subset of oligonucleotides using a primer pair, wherein a first primer of the primer pair binds to the universal primer binding site and a second primer of the primer pair binds to the target-specific primer binding site to produce the subpool of subject-specific oligonucleotides;
wherein the second primer is chosen based on the gene profile for the subject, and wherein the amplifying produces oligonucleotides with a content region comprising genes specified in the gene profile; thereby preparing the subpool of subject-specific oligonucleotides.

2. The method of claim 1, wherein the subject-specific oligonucleotides are target enrichment primers.

3. The method of claim 1, wherein the first primer or the second primer comprises a recognition element.

4. The method of claim 3, wherein the recognition element is biotin.

5. The method of any one of claims 1-4, wherein the content region comprises a site specific endonuclease.

6. The method of claim 5, further comprising digesting the subpool of subject-specific oligonucleotides with a DraI restriction endonuclease to produce an DraI-digested subpool.

7. The method of claim 6, further comprising digesting the digested subpool to produce a single stranded capture probe.

8. The method of any one of claims 1-7, wherein the gene profile is obtained from a subject sample.

9. The method of claim 8, wherein the subject sample is a solid biopsy.

10. The method of claim 8, wherein the subject sample is a liquid biopsy.

11. The method of claim 8, wherein the subject sample is a blood sample.

12. The method of any one of claims 1-7, wherein the gene profile is obtained from a subject database.

13. The method of any one of claims 1-7, wherein the gene profile comprises a portion of a gene.

14. The method of any one of claims 1-7, wherein the gene profile comprises at least one full gene.

15. The method of any one of claims 1-7, wherein the gene profile comprises at least one exon of a gene.

16. The method of any one of claims 1-7, wherein the gene profile comprises at least 2 genes.

17. The method of claim 12, wherein the database comprises gene profiles from a plurality of subjects.

18. The method of claim 1, wherein the first pool is present on a microarray.

19. The method of claim 1, wherein the plurality of oligonucleotides comprises DNA.

20. The method of claim 1, wherein the plurality of oligonucleotides comprises RNA.

21. The method of claim 1, wherein the amplifying is accomplished using polymerase chain reaction.

22. The method of claim 1, wherein the amplifying produces oligonucleotides spanning at least a portion of a gene.

23. The method of claim 1, wherein the amplifying produces oligonucleotides spanning at least one full gene.

24. The method of claim 1, wherein the amplifying produces oligonucleotides spanning at least one exon of a gene.

25. The method of claim 1, wherein the amplifying produces oligonucleotides spanning at least 2 genes.

26. The method of claim 1, wherein the gene profile comprises a gene selected from the group consisting of: BRCA1, BRCA2, BARDI, TP53, BRAF, Myc, Bcl-2, CDKN1β, NOTCH1, EGFR, FGFR1, FGFR2, FGFR3, HNF1A, JAK1, JAK2, JAK3, KIT, KRAS, MET, SRC, and any combination thereof.

27. The method of claim 1, wherein an oligonucleotide database is consulted to select the second primer of the primer pair.

28. The method of claim 27, wherein the oligonucleotide database comprises instructions for locating the second primer of the primer pair.

29. The method of claim 27, wherein the oligonucleotide database comprises the oligonucleotide sequence for the second primer of the primer pair.

30. A system comprising
a) a computer readable memory storing on an electronic storage device an oligonucleotide database, and
b) a computer processor,
wherein the computer processor is configured to access the oligonucleotide database and select a primer pair comprising a first primer and a second primer from the oligonucleotide database that is capable of amplifying each of a set of genes from a gene profile after the set of genes is input into the system.

31. A kit comprising a first oligonucleotide pool that comprises a plurality of oligonucleotides, wherein each oligonucleotide comprises:
a) a universal primer binding site, wherein each oligonucleotide of the plurality of oligonucleotides has the same universal primer binding site;
b) a content region comprising at least a portion of a gene, wherein each oligonucleotide of the plurality of oligonucleotides has a different content region, and wherein the plurality of genes comprises BRCA1, BRCA2, BARDI, TP53, BRAF, Myc, Bcl-2, CDKN1β, NOTCH1, EGFR, FGFR1, FGFR2, FGFR3, HNF1A, JAK1, JAK2, JAK3, KIT, KRAS, MET, and SRC; and
c) a target-specific primer binding site, wherein oligonucleotides comprising a content region with a portion of the same gene have the same target-specific primer binding site.

32. A method comprising:
a) obtaining the subpool of subject-specific oligonucleotides of any one of claims 1-29
b) contacting the subpool of subject-specific oligonucleotides with a subject sample; and
c) performing a sequencing reaction.

33. A method of monitoring the progression of a tumor, comprising:
a) obtaining a first pool of primers, wherein the first pool of primers comprises a plurality of oligonucleotides, and wherein each oligonucleotide comprises:
(i) a 5′ universal primer binding site, wherein each oligonucleotide of the plurality of oligonucleotides has the same 5′ universal primer binding site;
(ii) a content region comprising at least a portion of a gene, wherein each oligonucleotide of the plurality of oligonucleotides has a different content region, and wherein a subset of oligonucleotides within the plurality of oligonucleotides comprises a content region with at least a portion of a same gene and
(iii) a 3′ target-specific primer binding site;
b) amplifying exponentially from the first pool the subset of oligonucleotides using a primer pair, wherein a first primer of the primer pair binds to the 5′ universal primer binding site and a second primer of the primer pair binds to the 3′ target-specific primer binding site to produce the subpool of subject-specific oligonucleotides;
c) contacting polynucleotides isolated from a sample obtained from the subject with the subpool of subject-specific oligonucleotides;
d) performing a sequencing reaction; and
e) repeating steps (c)-(e) for a period of time, thereby monitoring the progression of the tumor.
